(19) United States
(12) Patent Application Publication
(10) Pub. No.: US 2022/0096371 A1
(43) Pub. Date: Mar. 31, 2022

(54) BIODEGRADABLE MICRONEEDLE SYSTEM WITH SUSTAINED RELEASE

(71) Applicant: University of Connecticut, Farmington, CT (US)

(72) Inventors: Thanh D. Nguyen, South Windsor, CT (US); Nicholas Farrell, Hebron, CT (US)

(21) Appl. No.: 17/487,661

(22) Filed: Sep. 28, 2021

Related U.S. Application Data
(60) Provisional application No. 63/084,211, filed on Sep. 28, 2020.

(51) Int. Cl. A61K 9/00; C08G 63/08; A61K 45/06

(52) U.S. Cl. CPC A61K 9/0021; A61K 45/06; C08G 63/08

(57) ABSTRACT

A transdermal, biodegradable microneedle system and method for treating pain in a patient. The transdermal easy-to-use biodegradable microneedle system can be fully embedded into the skin to perform a sustained and nearly zero-order release kinetics of therapeutics over a long period of time (at least 30 days) for the treatment of pain.

### CROSS-REFERENCE TO RELATED APPLICATIONS

This application is a non-provisional of and claims the benefit of U.S. Provisional Patent Application No. 63/084,211, filed on Sep. 28, 2020, the entire contents of which are incorporated herein by reference.

### BACKGROUND

Millions of Americans suffer from arthritis (e.g., rheumatoid arthritis and osteoarthritis), diseases associated with inflammation and extreme joint pain. The first treatment choice for these diseases is to use an analgesic or anti-inflammation drugs which can alleviate pain/inflammation and enable patients to regain normal daily activities. Despite the strong effects of opioids for pain relief, the drugs cause significant side effects of drug-addiction and abuse. The opioid crisis in the country is largely due to the overuse of this drug to treat pain. Therefore, non-opioid drugs including steroids (e.g., dexamethasone), non-opiate and non-steroid anti-inflammation drugs (NSAIDs) such as Aspirin (ASA or acetylsalicylic acid) and others (e.g., lidocaine as local anesthesia) become favorable choices and are considered to be the golden treatment for arthritis patients. Oral administration of these drugs such as NSAIDs unfortunately causes significant side-effects on the gastrointestinal (GI) tract (stomach bleeding, stomach ulcers with cancer risks etc.), which stem from direct contact between these drugs and gastric mucosa. Additionally, many drugs such as dexamethasone or NSAIDs have short lifetimes and some drugs (e.g., ASA) exhibit a low bioavailability in oral use, thereby requiring daily administrations with a large dose, making the side effects on the GI tract even more severe.

Local treatment by injecting non-opioid analgesics alone or with hydrogels into joints have been employed. Yet, the injected solutions/gels have low retention due to constant joint motion. Additionally, a large volume of the implanted gels potentially causes pain and immune reactions. Again, the short lifetime of these drugs requires repeated inconvenient and painful injections, also generating millions of needles/syringes, which pose a significant risk of disease infection and an environmental burden to dispose the biohazardous waste.

Alternatively, topical application of analgesics is convenient, causes no pain, and avoids common side effects of oral/injection delivery. Several topical NSAID gels including Voltaren®, Capsaisin®, Arthricream®, etc. have been clinically used for arthritis patients. Although offering an excellent patient compliance and avoiding the GI side effects, these gels struggle with a tremendous limitation of drug absorption, mainly due to the existence of a skin outer barrier, called the Stratum Corneum (SC), which prevents the topical NSAIDs from penetrating into the body and systematically accessing synovial fluid of joints.

### SUMMARY

Transdermal microneedles (MNs) have appeared as a powerful drug-delivery system (DDS) to combine benefits of injections and topical administrations. MNs can penetrate the SC to facilitate the intra-skin delivery and systematic absorption of various drugs. Tiny MNs avoid touching the nerve endings to tremendously reduce pain and can be self-administered by non-professionals, consequently increasing patient compliance. Most significantly, transdermal MNs avoid direct contact of NSAIDs (and other drugs) with the gastric mucosal layer. Therefore, NSAID (e.g., ASA) MNs, which can dramatically reduce side effects on the GI system, have attracted interest in recent years. However, all reported analgesic MNs can perform only either immediate burst-release or sustained-delivery for a few days, which then still requires undesired repeated administrations. In addition, common drug-formulation approaches for the available MNs rely on a matrix-based system which lacks a separate/systematic control over the release time and drug-dose while suffering from the problem of a big initial burst release.

The present disclosure provides a novel (trans)dermal easy-to-use biodegradable MN system which can be fully embedded into the skin to perform a sustained and nearly zero-order release kinetics of NSAIDs/steroid/other analgesics over a long period of time (at least 1 month) for the treatment of musculoskeletal pain. As described, the system includes a unique core-shell MN system which has a biodegradable shell of PLGA (Poly Lactic-co-Glycolic Acid) and a matrix-core of PLGA, containing ASA (Aspirin).

ASA is a typical NSAID, commonly used for pain relief and anti-inflammation. Oral pills of ASA are also frequently used to treat blood clot and prevent stroke in patients with cardiovascular diseases. As described below, the core-shell MNs only need a single-time administration to create a sustained release over a long and extendable period of time with a sufficient daily therapeutic dose of ASA for an effective analgesic effect. The MNs can provide a safe, effective means to treat various types of pain, and the MN system can create a significant impact, bolstering the global effort to eliminate the use of highly-addictive opioids for pain treatment.

A core-shell structure may be manufactured using Stamped Assembly of Polymer Layer (SEAL), which includes the method to create core-shell PLGA MNs (see, for example, U.S. Patent Application Publication No. 2019/0269895, which is incorporated herein by reference).

The SEAL method however only creates a delayed sharp burst release. It is not able to provide a sustained release. This matrix-based system provides a complicated release with a large initial burst and a mutual dependence of drug-dose and release time. The other sustained release MNs are based on the matrix-based system which has the drugs uniformly dispersed/distributed over a biodegradable matrix of hydrogel or PLGA. This matrix-based system provides a sustained release based on the degradation profile of the PLGA/hydrogel matrix. The release period and drug daily dosing are thus mutually dependent. To extend the release period, one needs to use a longer degradation matrix which then compromises the daily release dose. This matrix-based system is complex, requires extensive formulation and does not provide for systematic control, which makes it challenging to achieve a long release period (e.g., 1 month, 2 months, 3 months or 4 months or more) with a sufficient daily release dose.

Although analgesic MNs (such as NSAID MNs) have been extensively studied for transdermal delivery of ASA, there are no current MN systems which can provide a sustained-release with a systematic and independent control of the drug-dose and release time over a long period of time, such as a few days (e.g., 1-3 days). The available MN systems mainly rely on a conventional formulation approach which mixes the drug inside a biodegradable polymer or hydrogel matrix (i.e., matrix-based drug delivery system) to obtain a sustained release.

The MN system disclosed herein (made of the PLGA/drug matrix core and PLGA shell) overcomes all of these limitations to provide for the first time, the ability to control and achieve any long sustained release period (e.g., greater than 30 days) by combining matrix-based core and the reservoir-based system with delayed sustained release to achieve the goal. The MN system reduces the need for frequent administrations of many important drugs such as pain medicine, growth hormone, insulin, cancer chemotherapeutics, cancer immune-therapeutics, vaccines, thus significantly increasing patient compliance and reducing health care costs.

The MN system disclosed herein can be easily inserted into the skin to perform a unique nearly zero-order release kinetics. The MNs allow, for the first time, to extend the release period without compromising the daily drug-dose. The unique MNs employ a new 3D fabrication method which creates the MN shell and drug core from independent processes, consequently gaining an independent control over the release period and drug dose. Another novel feature is the combination of the matrix-based drug-delivery system (i.e., the MN matrix core to obtain a sustained delivery over a short period; e.g. ˜14 days) and reservoir-based system (i.e., the core-shell structure) to obtain a series of sequential sustained delivery (e.g. after 14 days, 28 days, etc.), consequently, extending the period of release without compromising the drug dosing. In brief, the use of an advanced 3D manufacturing process, a novel drug-delivery approach, and the achievement of a new core-shell MN structure with a unique delayed sustained-release provides a non-opioid long-acting analgesia with a single-time administration.

In one embodiment, the present disclosure provides a transdermal microneedle system comprising a substrate for application to skin, the substrate including a plurality of microneedles, each microneedle including a core and a shell, wherein the microneedles are configured to provide a sustained release of a therapeutic through the skin over a period of time with a near zero-order release profile.

In another embodiment, the present disclosure provides a transdermal microneedle system comprising a substrate including a plurality of microneedles, each microneedle including a core and a shell, the core including a therapeutic that when released through the shell provides a sustained release of the therapeutic into a patient over a period of time greater than 30 days to provide a daily therapeutic dose for an effective analgesic effect to the patient.

In yet another embodiment, the present disclosure provides a method of treating pain in a patient. The method comprises applying a transdermal microneedle assembly to the patient. The transdermal microneedle assembly includes a substrate and a plurality of microneedles coupled to the substrate, each microneedle including a core and a shell. The core includes a therapeutic, and wherein the therapeutic that when released through the shell provides a sustained release of the therapeutic into the patient over a period of time greater than 30 days to provide a daily therapeutic dose to reduce pain to the patient.

Other aspects of the invention will become apparent by consideration of the detailed description and accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a schematic of a transdermal microneedle assembly and zero-order sustained release of a therapeutic over a long period of time.

FIG. 2 illustrates a schematic of a microneedle structure of the transdermal microneedle assembly of FIG. 1 that provides a novel delayed sustained zero-order release.

FIG. 3 illustrates a typical nearly zero-order release from fabricated transdermal microneedle assemblies. Insert Images are representative core-shell MN patch and zoom-in MNs. The MNs are located on a supporting array before skin-insertion. Red dye is added to visualize the core. Graph illustrates the release of a drug model (Cur) from the MNs (Shell=PLGA50 15 KDa:30 KDa=1:1 blend, Core=Drug+PLGA50 30 KDa).

FIG. 4 illustrates preliminary data to show an independent control of delay time and drug dosing from the MNs. a. Increasing the molecular weight of PLGA of the shell and lactide component provides longer delays. b. Increasing drug-core concentrations can enhance release-dose per day (triplicate measurement/data and small error bars are invisible).

FIG. 5 illustrates extension of release time. Combining two MN sets; one has a sustained release from date 0-1434, 38, and the other has a release from date 14-30 to obtain 30-day release. To further extend the release, another MN set can be added that starts to release from date 31.

FIG. 6 illustrates the mechanical strength of the MNs. a. Force vs displacement (n=3 MN patches). b. Images of the MNs inserted into a porcine skin. c. SEM images of the MNs before and after human-skin insertion (immediately withdrawn) d. A histology slide (H&E) of the MN (stained by tattoo ink) in the human skin (scale bars=300 μm for c & d).

FIG. 7 illustrates the rapid healing of skin after MN insertion without irritation. a. Images of the skin show a rapid healing after MN insertion. b. Zoom-in images of the wound holes show a rapid healing (within 1 hour) to close up the wound and entrap the MNs inside the skin. Scalebars=300 μm.

FIG. 8 provides in vivo imaging (IVIS) to show different delay or lag times are obtained from the core-shell MNs.

FIG. 9 illustrates the efficacy of transferring MNs from the patch to skin after insertion.

FIG. 10 illustrates consistency and reproducibility of delayed-burst release of the MNs (containing drug core without PLGA) in different skin conditions (n=3).

FIG. 11 illustrates preliminary data on using the core-shell MNs for a sustained release of TAF over 28 days (patch size=1×1 cm2). The MNs #1 are only TAF/PLGA core without PLGA shell (TAF:PLGA=3:7; PLGA5050 30 kDa). The MNs #2 have the same core and a PLGA shell (PLGA 5050, 30 kDa) to provide the delayed sustained release after the release period of the MNs #1 (n=3 MN patches/data).

FIG. 12 illustrates the pH from degrading PLGA MNs modulated by the addition of MgO inside the MN PLGA drug cores

Before any embodiments of the disclosure are explained in detail, it is to be understood that the disclosure is not limited in its application to the details of construction and the arrangement of components set forth in the following description or illustrated in the following drawings. Also, it is to be understood that the phraseology and terminology used herein is for the purpose of description and should not be regarded as limiting.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not. Further, it should further be noted that the terms “first,” “second,” and the like herein do not denote any order, quantity, or relative importance, but rather are used to distinguish one element from another.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. All ranges disclosed herein are inclusive of the endpoints, and the endpoints are independently combinable with each other. Each range disclosed herein constitutes a disclosure of any point or sub-range lying within the disclosed range.

As used herein, the terms “providing”, “administering,” and “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, “treat,” “treating” and the like mean a slowing, stopping or reversing of progression of a disease or disorder when provided a composition described herein to an appropriate control subject. The terms also mean a reversing of the progression of such a disease or disorder to a point of eliminating or greatly reducing the cell proliferation. As such, “treating” means an application or administration of the compositions described herein to a subject, where the subject has a disease or a symptom of a disease, where the purpose is to cure, heal, alleviate, relieve, alter, remedy, ameliorate, improve or affect the disease or symptoms of the disease.

All documents cited herein and the following listed documents that are attached hereto for submission, all referenced publications cited therein, and the descriptions and information contained in these documents are expressly incorporated herein in their entirety to the same extent as if each document or cited publication was individually and expressly incorporated herein.

While the invention has been described with reference to preferred embodiments, it will be understood by those skilled in the art that various changes may be made and equivalents may be substituted for the elements thereof without departing from the scope of the invention. In addition, many modifications may be made to adapt the teaching of the invention to particular use, application, manufacturing conditions, use conditions, composition, medium, size, and/or materials without departing from the essential scope and spirit of the invention.

While various aspects and embodiments have been disclosed herein, other aspects and embodiments will be apparent to those skilled in the art. The various aspects and embodiments disclosed herein are for purposes of illustration and are not intended to be limiting of the true scope of the invention disclosed herein. It is also to be understood that the terminology used herein is for the purpose of describing particular embodiments only, and is not intended to be limiting. Since many modifications, variations, and changes in detail can be made to the described examples, it is intended that all matters in the preceding description and shown in the accompanying figures be interpreted as illustrative and not in a limiting sense.

All methods described herein can be performed in any suitable order unless otherwise indicated herein or otherwise clearly contradicted by context. The use of any and all examples, or exemplary language (e.g., “such as”), is intended merely to better illustrate the invention and does not pose a limitation on the scope of the invention or any embodiments unless otherwise claimed.

The present disclosure provides a transdermal easy-to-use biodegradable MN system which can be fully embedded into the skin to perform a sustained and nearly zero-order release kinetics of NSAIDs over a long period of time (at least 30 days) for the treatment of musculoskeletal pains. The present disclosure also provides a method of treating pain in a patient over a long period of time (at least 30 days) with a sustained release of a daily dose of a therapeutic and avoiding a burst release of the therapeutic. The method involves applying a transdermal microneedle assembly to the patient. The transdermal microneedle assembly includes a substrate, and a plurality of microneedles coupled to the substrate, each microneedle including a core and a shell. The core includes a therapeutic, wherein the therapeutic that when released through the shell provides a sustained release of the therapeutic into the patient over a period of time greater than 30 days to provide a daily therapeutic dose to reduce pain to the patient.

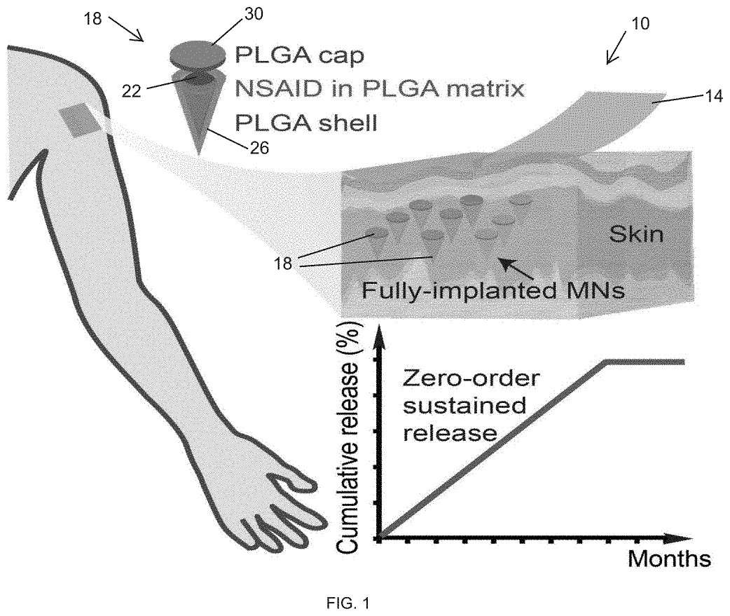

FIG. 1 illustrates the MN system 10 in accordance with an embodiment of the present disclosure. The MN system 10 include a substrate 14 and a plurality of microneedles 18 extending from the substrate 14. The plurality of microneedles 18 each include a core 22, a shell 26 surrounding the core 22, and a cap 30 to enclose the core 22. The same structure may be utilized for all or less than all of the microneedles 18.

The core 22 may comprise a therapeutic for application to a patient. The therapeutic may comprise pain medicine (e.g., NSAIDs, a steroid like Dexamethasone, local anesthesia like Lidocaine, and the like), chemotherapeutics, immunotherapeutics, small molecule drugs, and peptides (e.g., GLP-1), etc. The therapeutic may be mixed with a polymer, such as, for example, Poly Lactic-co-Glycolic Acid (PLGA) as a matrix. The core 22 of each microneedle 18 on a particular substrate 14 may comprise the same therapeutic. In other constructions, the core 22 of the plurality of microneedles 18 may vary and employ more than one type of therapeutic. The shell 26 comprises a polymer, such as, for example, PLGA and is biodegradable or a hydrogel matrix to obtain a sustained release.

The MN system 10 can be easily inserted into the skin to perform a unique nearly zero-order release kinetics. The MN system 10 provides an extension of the release period without compromising the daily therapeutic dose. The MN system 10 is utilizes a new 3D fabrication method which creates the shell 26 and core 22 from independent processes, thereby achieving independent control over the release period and therapeutic dose. Another novel feature is the combination of the matrix-based drug-delivery system (i.e., the MN matrix core to obtain a sustained delivery over a short period; e.g. ˜14 days) and the reservoir-based system (i.e., the core-shell structure) to obtain a series of sequential sustained delivery (e.g., after 14, 28 days, etc.), consequently, extending the period of release without compromising the therapeutic dose.

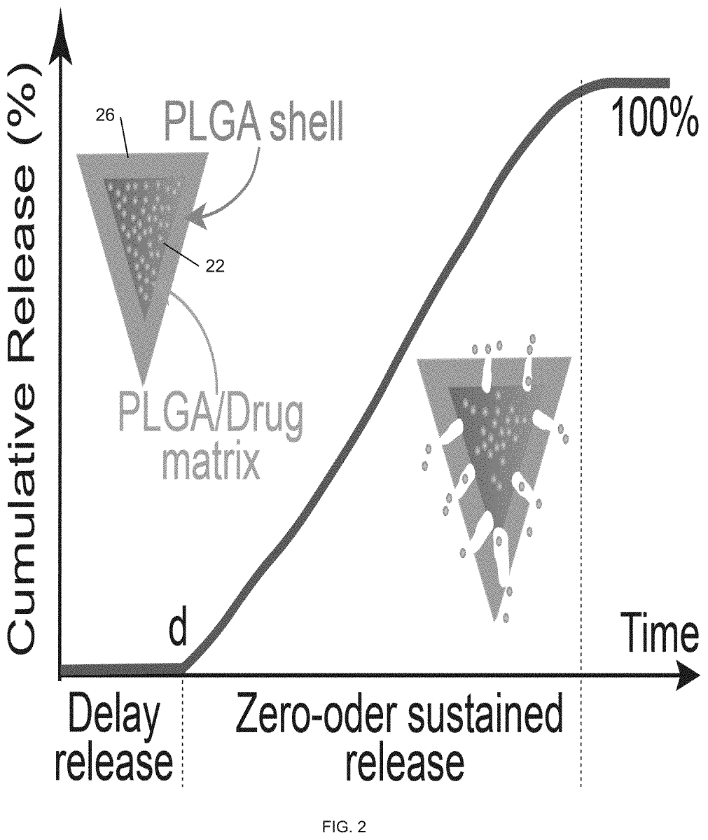

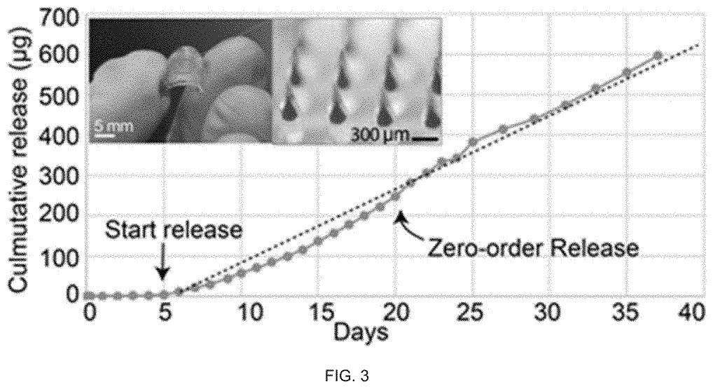

The structure and composition of the MN system 10 provides a sustained delivery of a therapeutic dose with a separate control of the therapeutic dose and a release duration to achieve an analgesic effect over an extended period of time (e.g., 15 or more days, 30 or more days, 45 or more days, 60 or more days). It is noted that several attempts have been made to create zero-order release kinetics to obtain an ideal release for a safe, effective drug-dose. There has been development of a biodegradable system relying on membrane-controlled reservoir structures to provide zero-order kinetics. However, all of these systems are large and need invasive implantation surgery. So far, there has not been any success to achieve small-scale MNs which can be easily inserted into skin, even by patients, to perform the zero-order release profile. In contrast, in the MN system 10 as disclosed herein, when the drug releases due to the formation of small pores on the PLGA degrading shell 26, the gradual depletion of the drug from the PLGA-matrix core creates a nearly constant release-rate (i.e., zero-order release), as seen in FIGS. 2-3.

### EXAMPLES

### Example 1: MN System with Aspirin as Therapeutic

Overall target dosing and release profile: ASA (aspirin) has a low bioavailability (˜20%) and can be used with an oral dose of ˜20-80 mg/day. Because the drug has a short half-life time, the oral form needs a large dose to sustain the plasma dose. The body clearance rate is also proportional to the initial amount of drug present inside the blood. Therefore, a sustained-delivery MN system, which gradually releases a small dose, is expected to require much less drug amount. As an example, a sustained-release MN patch provides a steady plasma dose for 14 days while an injection of the same drug (i.e., immediate release), is rapidly cleared out in only 2 days. Taking into account of 20% bioavailability, short life-time, and the dose-benefit of sustained release, the MN system 10 is targeted to obtain a daily ASA dose of about 30 times (roughly ˜5 times of bioavailability×6-7 times from sustained release) less than the oral pills (˜81 mg). This results in a release rate of at least 81:30˜2.7 mg/day in vitro. For in vivo, the MN system 10 is targeted to obtain an average daily plasma concentration of ˜100 ng/ml and ACU (area under the curve) of ˜865 ng/ml.hr, equivalent to those obtained from an 81 mg ASA oral pill, identified by HPLC (High-performance liquid chromatography). As an example, the MN system 10 is targeted to achieve a sustained release of at least 30 days which has not been achieved for any reported NSAID/ASA MNs. A longer release period (e.g., 2-3 months) (if needed) can be obtained by the controlled-delivery approach (described below).

Drug stability: ASA is a small molecule and very stable at high temperature (Tmelt˜1350 C). Thus, the drug will stay active for a long-term delivery inside the body. The stability will be re-assessed and affirmed during the study of ASA release, using HPLC (High Performance Liquid Chromatography).

### Example 2: Characterizing the Release Kinetics of the Core-Shell MNs and Engineering Parameters of the Release Profile (i.e. Dose, Delay/Lag Time, and Release Period) In Vitro

Preliminary data: Achieving the core-shell MNs with nearly zero-order release kinetics; As shown in FIG. 3, an example of the core-shell MN set has a PLGA shell which is a blend of PLGA5050 15 KDa and 30 KDa (1:1). The core is a matrix of PLGA5050 30 KDa and Curcumin (Cur), a drug model, which has a similar hydrophobicity of ASA and can be easily quantified, using UV-microplate reader. In a buffer solution to simulate skin-fluid (PBS+25% ethanol), these MNs as an example do not release for 5 days and then start a nearly zero-order release kinetics over 1 month.

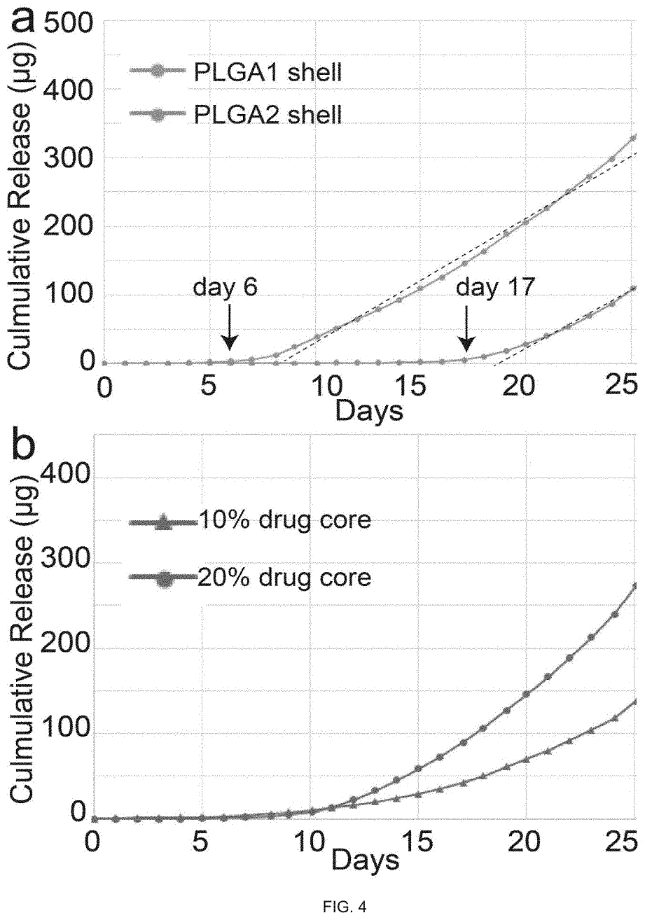

Controlling lag/delay time: Any other delay time (e.g. 14, 28, days etc.) can be obtained by using different MN PLGA shells as shown in FIG. 4 (at a). MNs with PLGA-2 shells (PLGA 5050 60 kDa) start to release after 14-16 days while MNs with PLGA 1 shells (PLGA 5050 15 kDa blended with 5050 30 KDa) quickly start to release after 5 days. Thus, increasing molecular weight or lactide component of the PLGA shells will increase the lag/delay time. The polymers can be blended together to obtain a lag time in between. This control of the lag-time from a few days to months has been also illustrated in our previous work for core-shell PLGA microparticles. Note that as the therapeutic cores are the same, the two MN sets do not exhibit much difference in the therapeutic daily-dose (i.e., similar slopes from the two straight lines in FIG. 4 (at a), illustrating a systematic control over the lag time (independent from drug-dose). Consistency and reproducibility of the release were also preliminarily demonstrated.

Controlling and scaling up the drug dose; FIG. 4 (at b) shows the two MN patches with the same PLGA shell and loaded with different drug concentrations (10% and 20%) provide two different daily doses. The MNs with 20% drug core provide a dose of ˜30 μg/day, about two times of that obtained from the MNs with 10% drug core. Despite the difference of release doses, both of the MN sets provide the same delay/lag time (˜11 days) due to the same PLGA shell, showing the excellent control over the release rate (independent from the delay time).

Scaling up the dose: Given such a 30 μg/day release as an example from one patch (0.5×0.5 cm2), a bigger patch (˜5×5 cm2) can be used which is ˜100 times of the current patch to obtain a very large dose of 30×100˜3 mg/day as desired. Such a large patch has been tested in patients for self-administration. Additionally, the MN density and/or size of the therapeutic-core can be increased to further enhance the therapeutic dose. Oral pills with much less dose can also be used to compensate the ASA MNs (if needed).

Microneedle design; the microneedles include a height of 600 μm and diameter at the base of 300 μm (FIG. 3). The MNs are positioned on top of a supporting PLA array (200-600 μm high). These sizes are common for MNs which have been previously tested for human use. The therapeutic-core is 200 μm (diameter) and 300 μm high. For rats (in the in vivo model), which have a dermal layer of about 1200 μm thick, and 400-2000 μm deep from the epidermis, the design allows the entire MNs to be fully embedded inside the dermis. Note that the MN dimensions can be modified by changing the MN molds.

Fabrication of the MNs: ASA was mixed with PLGA inside Acetone or Ethanol and deposited on a Teflon film for drying inside a vacuum-assisted desiccator to form a solid film (with different ASA concentrations). The film can also be lyophilized for a complete removal of the solvent. A pre-fabricated silicone or Polydimethylsiloxane (PDMS) molds were employed to fabricate the MN shell, core and cap. These components were assembled together to form the MNs, following the SEAL (StampEd Assembly of polymer Layer) method, previously described in U.S. Patent Application Publication No. 2019/0269895, which is incorporated herein by reference. In some constructions, a hydrophilic layer of PVP (polyvinylpyrrolidone) was coated onto the microneedle core before inserting the microneedle PLGA and therapeutic core into the PLGA shell. This step is important to avoid the deformation of the core when the engaged core/shell systems are heated for bonding them together under the heat. The PVP can be drop-casted from a water-based solution onto the microneedle core after the fabrication process.

To assess stability of ASA: the ASA was prepared inside different buffer solutions, including PBS (phosphate buffer solution)+ethanol of 0-25% (to mimic interstitial skin-fluid) and different simulated body fluids (following reported formulations) at 370 C. Once a week for a total of 2 months, aliquots from the samples were collected and the active ASA was quantified by comparing the HPLC's retention time to that obtained from freshly made ASA (see methods described above). To assess release kinetics, the ASA MNs were placed inside different buffer mediums (as described above). Over the course of two months, the supernatant from the samples was collected to assess the daily-released ASA, using HPLC. As skin could be subjected to temperature changes, the MNs, conditioned at a wide temperature range of 28-370069-71 in vitro were reassessed.

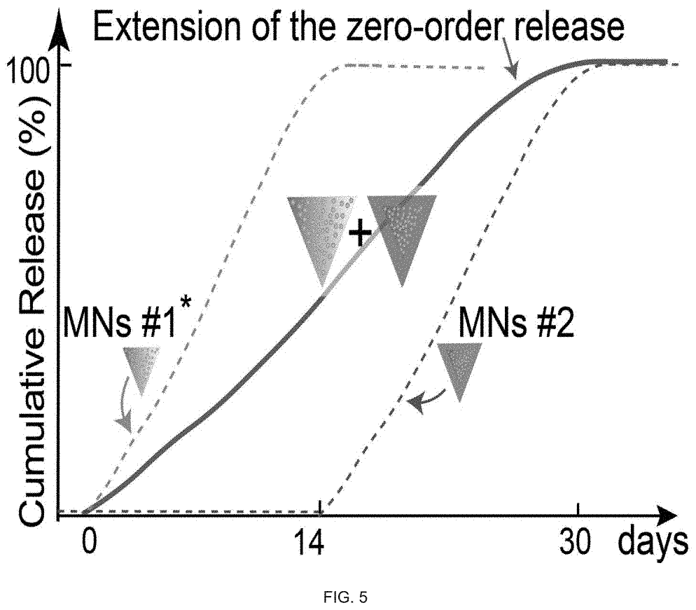

Controlling release kinetics and therapeutic-dosing: Controlling the delay time: the delay or lag time of the MNs can be tuned by changing the PLGA shell as shown in FIG. 4 (at a). As such we will construct a library of varying lag times with different PLGA shells. Controlling therapeutic-dosing: the amount of therapeutic released per day can be controlled by fabricating the core with different drug concentrations (see above and FIG. 4 (at b). Alternatively, the MN density/core can be increased or multiple MN patches can be applied, which virtually cause no pain. A large MN patch (5×5 cm²) which has been tested in human can also be applied. Controlling the release period: the unique delayed release of the core-shell MN system 10 can be combined with different MNs to create a consecutive release over a long period without affecting the released dose. FIG. 5 illustrates an example of this approach where the reported matrix-based MN formulation, which only has the drug/PLGA-matrix core without the PLGA shell, can be adapted to obtain a sustained release from day 0 to day 14. This MN set (MNs #1) can be combined with the core-shell MNs (MNs #2) which start to release on day 15 and has the same matrix core as the MNs #1 to obtain another 14-day sustained release, leading to a total of ˜30 days release. If the release needs to be extended further, another MN set which starts to release on day 31 can be added and thus, the release time can be continually extended without compromising the release dose. The preliminary data shown in FIG. 4 (at b) and described above along with the previous work have shown a highly controllable and consistent delay time, obtained from the core-shell PLGA structure. An assembly of small MN patches (with different delay times) onto a single large patch has been also demonstrated in clinical trials.

Assessment of mechanical properties: the microneedles were sandwiched between two glue-coated clamps of an Instron tensile machine. The MNs were axially compressed and sheared by the machine with a constant strain rate. The maximal stresses, which break the MNs, (i.e., failure force) were recorded and statistically compared to those of a homogeneous PLGA film, as reported.

The MNs will provide a release for at least 30 days with >3 mg/day in vitro. About ten formulations of the MNs fulfilling these release criteria and having a sufficient strength (failure force >0.07 N/m for human-skin penetration) will be used for in vivo study. The scalability might be limited by the photolithograpy-based fabrication of the MN molds. High-resolution 3D-printers can be employed to create the molds, significantly scaling up the fabrication for mass production. If multiple drugs are needed, we can formulate them into the same PLGA core-matrix of our MNs when these drugs are soluble in the same solvent (e.g., Cur and ASA are both soluble in ethanol). Otherwise, an emulsified PLGA/drug nano/microparticles can be used inside the MN cores for the sustained release. Alternatively, the assembly method presented here provides the ability to make multiple drug-compartments in the MN core for co-delivery.

### Example 3: Assessing the In Vivo Release and Demonstrating the Prolonged Analgesic Effect of the MNs

The MNs sustain a plasma therapeutic dose of ASA and provide a prolonged analgesic effect, superior to the use of topical NSAID gels and similar to the effect of daily injection.

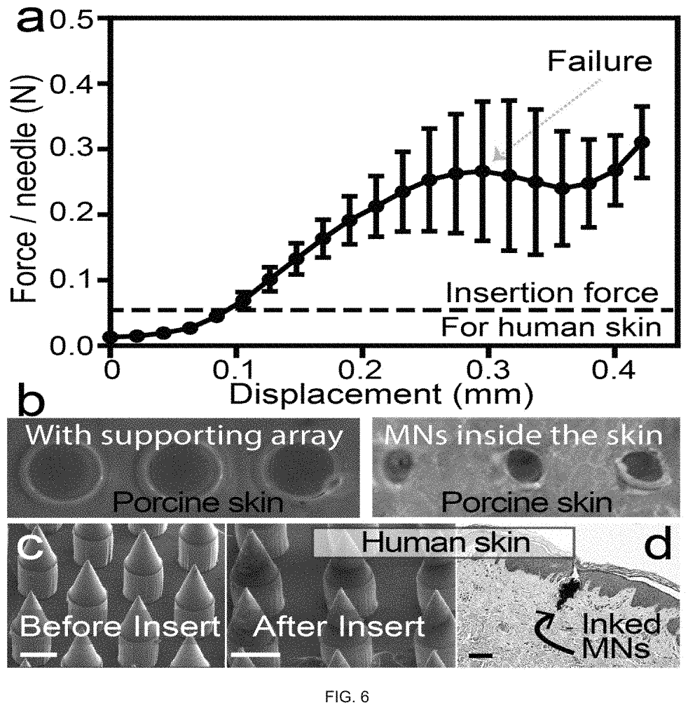

Preliminary data: MN strength for human-skin insertion: the strength of the softest PLGA MNs, made with the shell of PLGA 5050 15 kDa and the PLGA 5050 30 kDa 1:1 blend were tested (the same core was used for all formulations). An eXpert 5952F tester (ADMET, USA) was employed to apply compression on the MNs. As shown in FIG. 6 (at a), the MNs appeared to fail at ˜0.3 N/needle, much higher than the threshold (˜0.07 N/needle). Furthermore, these MNs were inserted into porcine skin, a well-known model for human skin (FIG. 6 (at b)). Importantly, when inserting into a fresh explanted human-skin (Extherid Bioscience Inc.), the MNs remain a good shape, as seen in SEM images of FIG. 6 (at c). Histology image (MNs stained with tattoo ink) in FIG. 6 (at d) shows that the MNs stay inside the dermis of the human-skin. Thus, the MNs will have sufficient strength for the skin insertion.

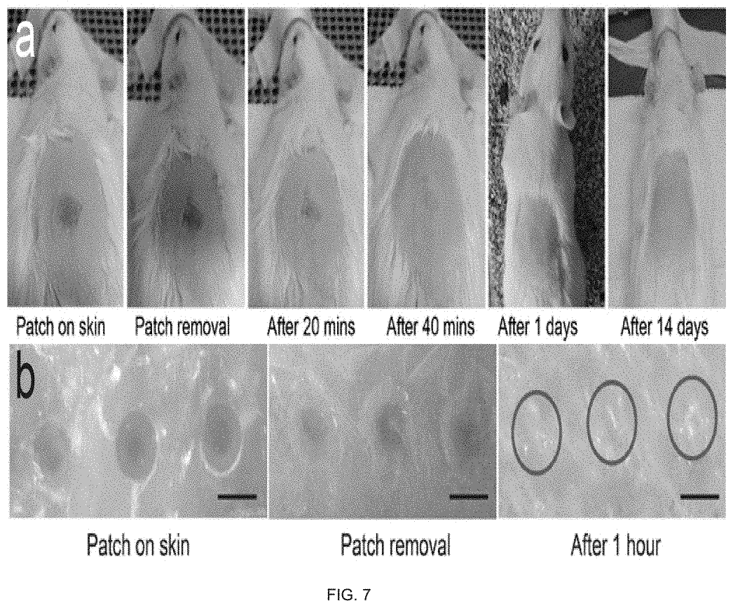

Rapid healing of skin after insertion with no irritation: Using a commercial MN applicator (Micropoint Technologies Pte Ltd), the MNs were easily inserted into rat skin. The supporting array, coated with a sacrificial layer of water-soluble PVP (polyvinylpyrrolidone), can be easily separated from the MNs. FIG. 7 shows the tiny wound holes (˜300 μm) can be quickly healed (<1 hour) to encapsulate the MNs inside the skin without skin damage or irritation.

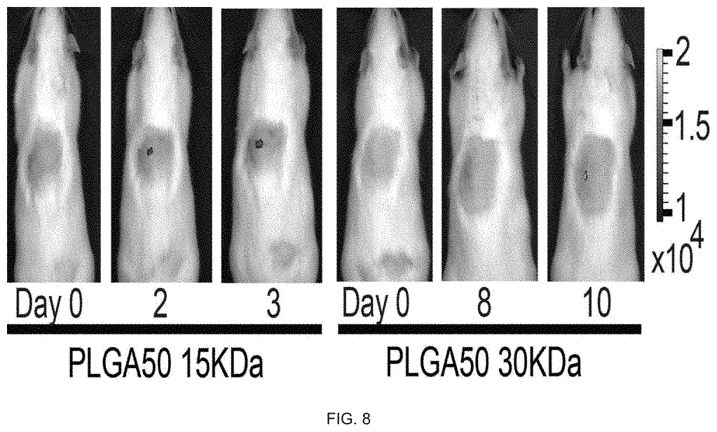

Controlling lag-time in vivo: the MNs were fabricated with the core containing a red dye of Rhodamine B. The MNs were inserted into rat skin. The dye was only visible when it is released out. FIG. 8 shows that the MNs made of PLGA shell with longer degradation provide a longer delay time. These together show the MNs can be easily inserted into the skin to perform the desired release kinetics.

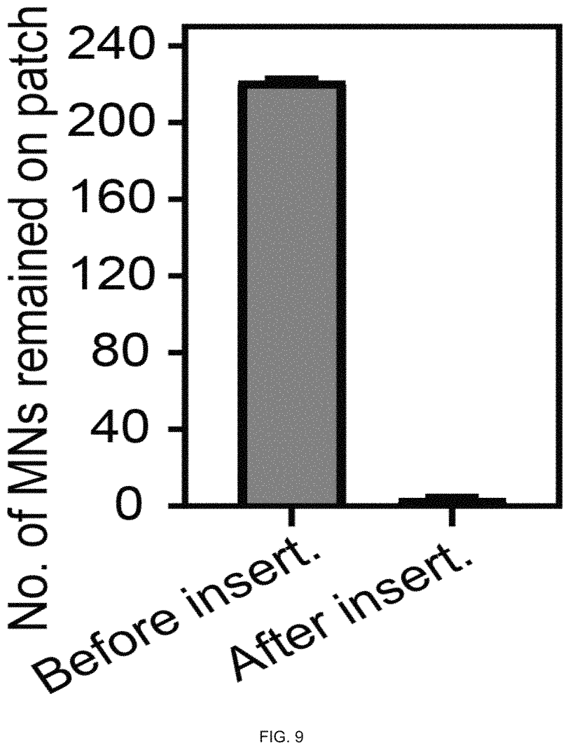

MN-Transferring efficiency: the number of MNs (the softest MN design) remained on the supporting array were quantified before and after insertion. As seen in FIG. 9, 97% of the MNs for all patches (n=3) were able to be penetrated into the rat skins, showing a high efficiency of the skin-administration and affirming the strength of the MNs for an effective skin-insertion.

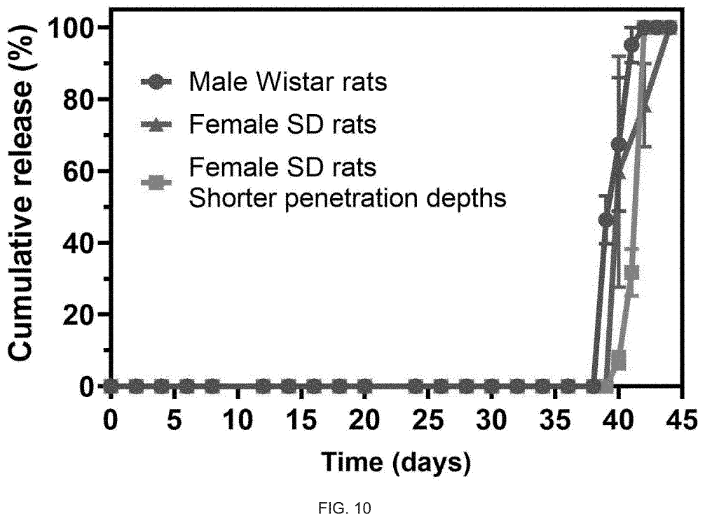

Consistency of the MN release in different skin conditions: To preliminarily test the consistence of drug-release, the delay-times from the MN sets which only contain drugs in the core without the PLGA-matrix (i.e., they just exhibit a delayed sharp burst-release) were assessed. Female SD rats and male Wistar rats were used for different skin conditions. In addition, different supporting arrays were employed to insert the MNs into different skin depths. As shown in FIG. 10 of quantified IVIS data, a consistent release (˜40 days) was obtained using the same MN design (same PLGA 7525 80 kDa shell). Thus, the MNs provide a reproducible/consistent release kinetics, affirming a quality of the MNs, also from batch-to-batch of the fabrication process.

Assessing pharmacokinetics (PK) of ASA MNs: the lead MNs described above were used to study release kinetics in vivo. Sprague Dawley (SD) rats, both male and female were utilized. The MNs were inserted into the animal back and the body fluid was allowed to dissolve the sacrificial PVP layer of the supporting array for ˜5 minutes for needle separation. The tiny holes on the animal skin were quickly closed to entrap the MNs inside the skin (see FIG. 7). The experiment was designed with four animal groups (n=6 rats/group, see statistical analysis) with an endpoint at 3 months (see table 1).

TABLE 1
Design for the study of in vivo release
and the use of ASA MNs for pain treatment

| | |
|---|---|
| Group 1 (exp.) | MNs loaded with ASA |
| Group 2 (control) | MNs without ASA |
| Group 3 (control) | Daily injections of the same ASA |
| Group 4 | Topical salicylate gel (Arthricream ® ~30 gram/day |
| | for 3 mg salicylate/day) |

Blood from the animals were collected three times a week (see vertebrate animal) for HPLC assessment. Synovial fluid was collected once a week to minimize damage on the rat's knee joint. The method to collect synovial fluid was previously reported. Briefly, a two-needle system (23 gauge and 25 gauge) was used to perform perfusion on the fluid. From the 23-gauge needle, the tubing was connected to a syringe pump to generate a saline perfusion (100 μl/ml). Both of these needles were punched into the knee joint of anesthetized animals. 250 μl of synovial fluid was collected from the outlet of the 25-gauge needle. The use of HPLC to quantify ASA is well-established. Briefly, a calibration curve was constructed with known amounts of ASA. A commercial PR18 column and a mobile phase rate of 1.5 ml/min were used. Retention time and optical reading (at 280 nm) were recorded. The AUC, compared to the standard curve to quantify the ASA was calculated. The HPLC was run on blank blood and synovial fluid to remove the background noise. Finally, non-compartmental analysis (using a software of Phoenix WinNonlin, Pharsight Certara. Inc) was employed to identify other PK parameters (Cmax, Tmax, and half-life t1/2).

To assess skin irritation (if any), the same animals in table 1 were assessed by non-invasive methods of Draize scoring and trans-epidermal water loss (TEWL)88-90. Briefly, to measure the skin's irritation, viewers were employed to grade skin's parameters in a blind manner. Score 0-4 presents the redness/swelling and necrosis from the lowest to the highest level. The number was averaged and the ratio of the scores between administration site and control site were compared between experimental and control groups. To measure TEWL, a Tewameter (Richmond Scientific) was used to obtain the reading for skin-dryness at the administration site. The numbers were then compared between the experimental and control groups. Using ELIZA, inflammation markers (e.g. IL-1α, TNF-α) in the collected blood were quantified, following given protocols.

In vivo pain model: a nerve constriction injury (CCI) model was used for chronic pain, established in the lab, to demonstrate the analgesic efficacy of the ASA MNs. It is well known that aspirin-triggered lipoxin (ATL) inhibits the spinal JAK2/STAT3 signaling, and thus also attenuate neuropathic pains. Indeed, the CCI animal model has been extensively used to test the analgesic efficacy of ASA.

Assessing analgesic-efficacy: As female rats are more susceptible to pain, female SD rats were used for this study. Briefly, the rat sciatic nerve was exposed and loosely tightened by 4-0 vicryl sutures. The MNs were then inserted into the rat skin. There were 4 groups (n=6 rats/group), similar to table 1. To assess analgesic efficacy, the Von Frey method (mechanical allodynia) was used and paw withdraw latency under thermal hyperalgesia to quantify the pain level (see details in vertebrate animals). Briefly, in Von Frey method, each rat was placed beneath an inverted plastic box with an elevated wire mesh bottom. From below the mesh, a series of calibrated von Frey filaments were applied to the mid-plantar surface of the hind paw. The peak of force in grams was recorded with a cutoff value at 100 g. In each animal, triplicates of each hind paw were taken. Data was analyzed using the up and down method of Dixon and Mood. The response was considered positive if the animal exhibits any nocifensive behaviors including brisk paw withdrawal, licking or shaking of the paw. Stimulus independent nociception was evaluated by following the dynamic weight bearing behavior (Bioseb device). The rats were allowed to move freely and the pressure data and live video were recorded by the system which also enables the analysis of the paw weight distribution and paw print area. For thermal hyperalgesia: Sensory blockade was evaluated by withdrawal latency to noxious heat using a paw thermal stimulator. The baseline latencies were measured on the right hind paw just before surgery (0 h) and post-surgery at pre-determined time points based on preliminary studies (6 h, 12 h, 1-60 days, once every two days). For each measurement, the animal was briefly placed on a hot surface, and the latency to the first licking/lifting of the left hind paw in response to the heat was recorded. Three independent observers recorded the latencies. Increased TWL latency and therefore decreased sensitivity, was interpreted as a reduction in thermal hyperalgesia. Difference of latency between groups at each time point was compared (see statistical analysis).

The single time administration of the MNs provided a sufficient therapeutic daily-dose of ASA inside blood (>100 ng/ml) for at least 1 month. Furthermore, the ASA MNs provided a prolonged analgesic effect, superior to the use of topical gels and similar to that obtained from daily injections of ASA. In case of a higher ASA plasma dose required to completely suppress pain, other MNs could be selected with higher release rate and re-assess the in vivo kinetics to have a much higher daily dose of plasma ASA in vivo.

Statistical Analysis: statistical comparisons between two groups was performed with two-tail student paired t-test (or ANNOVA with more than 2 groups) and log-rank test with a significance level of p=0.05. To identify the number of animal samples, power analysis and the common software of Gpower was used for comparison between means of two independent groups. A common power of 0.8 was input, a significance level of 0.05 and an effective size of d=1.8, which is defined by previous results, preliminary data and similar studies. There were 6 animals/group.

### Example 4: Sequential Therapeutic Releases from Different Microneedle Designs

An experiment was performed to show that the sequential releases from different microneedle designs (using different PLGA shells) to obtain different delayed times which then add up into a sustained nearly-zero order release for over a long period (>1 month) as seen in the data below (in this case; TAF or Tenofovir alafenamide was used as a drug model).

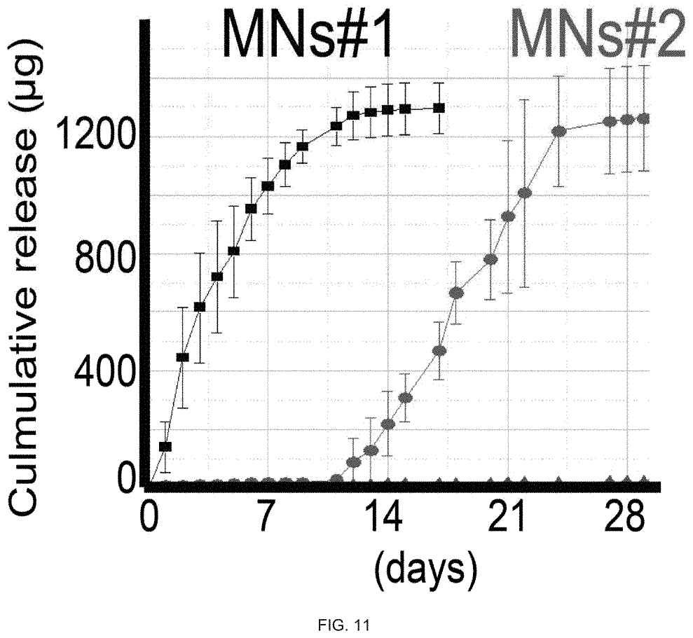

Using TAF as a drug model, the delayed sustained release from the core-shell MNs was obtained, similar to the Cur drug model described above. Two MN sets of TAF were fabricated with different release periods and combined together to achieve a longer sustained release (FIG. 11).

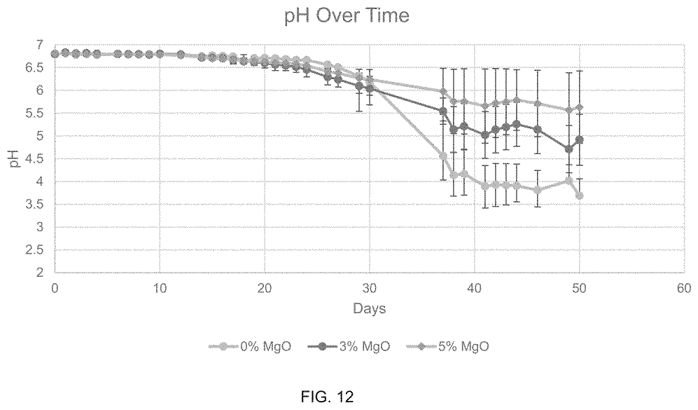

It was further discovered that MgO can be added into the PLGA to neutralize its acidic byproducts which could help to reduce the instability of drug caused by this acidic (low pH) environment from PLGA Degradation. The MNs were fabricated with PLGA 5050 30 kDa+TAF core and conditioned them inside a buffer which also has 7527PLGA shell to mimic the MN shell. As illustrated in FIG. 12, the pH over time with the MN cores added with different MgO concentrations was observed over time. As seen, the 5% MgO increases the pH from the PLGA and thus neutralizes the acidic byproducts of the PLGA degradation. This helps to significantly stabilize the drugs loaded inside the MNs.

Various features and advantages of certain embodiments are set forth in the following claims.

What is claimed is:

1. A transdermal microneedle system comprising:
a substrate for application to skin, the substrate including a plurality of microneedles, each microneedle including a core and a shell, wherein the microneedles are configured to provide a sustained release of a therapeutic through the skin over a period of time with a near zero-order release profile.

2. The transdermal microneedle system of claim 1, wherein the period of time is at least one month.

3. The transdermal microneedle system of claim 1, wherein the period of time is at least two months.

4. The transdermal microneedle system of claim 1, wherein the release of the therapeutic avoids a burst release.

5. The transdermal microneedle system of claim 1, wherein the core includes a first composition, and the shell includes a second composition, and wherein the first composition and the second composition are independently controlled to achieve the near zero-order release profile.

6. The transdermal microneedle system of claim 1, wherein the substrate includes a plurality of sections, and wherein the microneedles in a first section include a core and a shell configured to provide a sustained release over a first portion of the period of time, and wherein the microneedles in a second section include a core and a shell configured to provide a sustained release over a second portion of the period of time.

7. The transdermal microneedle system of claim 1, wherein the therapeutic is a pain reliever a chemotherapeutic, an immunotherapeutic, a small molecule drug, or a peptide.

8. The transdermal microneedle system of claim 7, wherein the pain reliever is aspirin.

9. The transdermal microneedle system of claim 1, wherein the core comprises a matrix of the therapeutic and a material comprising the shell.

10. The transdermal microneedle system of claim 9, wherein the therapeutic is a pain reliever and the material is PLGA.

11. The transdermal microneedle system of claim 1, wherein a first set of the plurality of microneedles includes a shell with a first composition and a second set of the plurality of microneedles includes a shell with a second composition, and wherein the shells with the first composition release their cores at a time different that the shells with the second composition to provide the sustained release.

12. The transdermal microneedle system of claim 1, wherein the sustained release provides a daily therapeutic dose for an effective analgesic effect to the patient.

13. A transdermal microneedle system comprising:
a substrate including a plurality of microneedles, each microneedle including a core and a shell, the core including a therapeutic that when released through the shell provides a sustained release of the therapeutic into a patient over a period of time greater than 30 days to provide a daily therapeutic dose for an effective analgesic effect to the patient.

14. The transdermal microneedle system of claim 13, wherein the shell and the core comprise a polymer.

15. The transdermal microneedle system of claim 14, wherein the polymer comprises PLGA.

16. The transdermal microneedle system of claim 13, wherein the therapeutic is a pain reliever a chemotherapeutic, an immunotherapeutic, a small molecule drug, or a peptide. pain reliever.

17. A method of treating pain in a patient, the method comprising:
applying a transdermal microneedle assembly to the patient, the transdermal microneedle assembly including
a substrate,
a plurality of microneedles coupled to the substrate, each microneedle including a core and a shell, the core including a therapeutic,
wherein the therapeutic that when released through the shell provides a sustained release of the therapeutic into the patient over a period of time greater than 30 days to provide a daily therapeutic dose to reduce pain to the patient.

18. The method of claim 17, wherein the substrate and the plurality of microneedles are biodegradable to avoid a removal process of the transdermal microneedle assembly.

19. The method of claim 17, wherein the shell and the core are configured to avoid a burst release of the therapeutic.

20. The method of claim 19, wherein the therapeutic is released through the shell with a near zero-order release profile.
